(19) United States
(12) Patent Application Publication
(10) Pub. No.: US 2009/0062615 A1
(43) Pub. Date: Mar. 5, 2009

(54) ENDOSCOPE WITH FOCAL LENGTH VARYING FUNCTION

(76) Inventor: Koji YAMAYA, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/127,479

(22) Filed: May 27, 2008

(30) Foreign Application Priority Data
Aug. 31, 2007 (JP) 2007-225908

(51) Int. Cl. A61B 1/002; G02B 15/14

(52) U.S. Cl. 600/167; 359/703

(57) ABSTRACT

The invention provides an endoscope with a focal length varying function, which includes an observation window, an objective optical system which is disposed in the observation window and is capable of varying a focal length thereof, and an endo-therapy product raising base which is oscillatable between a stored position where the endo-therapy product raising base is stored in the distal end section body (endo-therapy product projection port) and a raised position where the endo-therapy product raising base is projected from the distal end section body in accordance with a raising operation on a proximal side, wherein at a time of a state in which the endo-therapy product raising base is projected from the distal end section body by a predetermined amount, a predetermined part of the endo-therapy product raising base is set to approximately correspond to a best focus position of the objective optical system.

### CROSS-REFERENCE TO RELATED APPLICATIONS

This application is based upon and claims the benefit of priority from prior Japanese Patent Application No. 2007-225908, filed Aug. 31, 2007, the entire contents of which are incorporated herein by reference.

### BACKGROUND OF THE INVENTION

1. Field of the Invention

The present invention relates to an endoscope with a focal length varying function, which includes an objective optical system which can vary a focal length.

2. Description of the Related Art

In general, in an endoscope, the focal length of an objective optical system needs to be increased in order to perform enlargement observation of, e.g. a surface of a mucous membrane, which is a part to be observed in a body cavity. However, in order to guide (insert) the endoscope to the surface of the mucous membrane, a wide viewing angle needs to be secured. For this purpose, a zoom endoscope having an objective optical system with a variable focal length has widely been used.

If the focal length of the objective optical system increases, the depth of focus reduces. Consequently, the surface of the mucous membrane tends to be displaced from the best focus position of the objective optical system. In other words, the distance between the objective optical system and the surface of the mucous membrane tends to deviate from the best focus position of the objective optical system. As a result, an image of the surface of the mucous membrane, which is photographed, becomes out of focus. To cope with this problem, in the above-described zoom endoscope, a distal end hood is attached to a distal end portion of an insertion section of the endoscope. Thereby, in order to perform, at least, enlargement observation, the distance between the objective optical system and the surface of the mucous membrane is securely set at a desired value in advance so as to correspond to the best focus distance. A wide viewing angle is secured. Specifically, the distal end hood is a dedicated structural member for making the best focal length equal to the distance between the objective optical system and the surface of the mucous membrane, and for securing a wide viewing angle.

Jpn. Pat. Appln. KOKAI Publication No. H11-342104, for instance, discloses a zoom endoscope wherein the above-described hood, which is advancible/retreatable to associate with the variation of the focal length of the objective optical system, is provided around the distal end of the insertion section.

### BRIEF SUMMARY OF THE INVENTION

In consideration of the above, the present invention provides an endoscope with a focal length varying function, which includes an endo-therapy product raising base and an objective optical system with a variable focal length, wherein a distance between a part to be observed at a time of enlargement observation and the objective optical system can easily be made equal to a best focal length without using a dedicated structural member.

According to the present invention, there is provided an endoscope with a focal length varying function, comprising: an observation window which is disposed on a side of a distal end section of an insertion section; an objective optical system which is built in the observation window and is capable of varying a focal length thereof; and an endo-therapy product raising base which is disposed in a vicinity of an endo-therapy product projection port of the distal end section and is oscillatable between a stored position where the endo-therapy product raising base is stored in the distal end section and a raised position where the endo-therapy product raising base is projected from the distal end section in accordance with a raising operation on a proximal side, wherein at a time of a state in which the endo-therapy product raising base is projected from the distal end section side by a predetermined amount, a predetermined part of the endo-therapy product raising base is set to approximately correspond to a best focus position of the objective optical system.

According to the present invention, there is also provided an endoscope with a focal length varying function, comprising: a distal end section of an insertion section which is inserted in a body cavity; an observation window which is disposed on the distal end section and configured to observe a part to observed, which is located in the body cavity; an objective optical system which is built in the observation window and is capable of varying a focal length thereof at a time of observing the part to be observed; an endo-therapy product projection port which is disposed in a vicinity of the observation window in the distal end section and configured to project an endo-therapy product which is passed through the insertion section; and an endo-therapy product raising base which is disposed in the endo-therapy product projection port, is oscillatable between a stored position where the endo-therapy product raising base is stored in the distal end section and a raised position where the endo-therapy product raising base is projected from the distal end section in accordance with a raising operation on a proximal side, and raises the endo-therapy product when the endo-therapy product raising base is disposed in the stored position, thereby projecting the endo-therapy product from the endo-therapy product projection port, wherein when the endo-therapy product raising base is raised and projected from the distal end section by a desired length, the length is set to approximately correspond to a best focal length of the objective optical system.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a plan view of a distal end section of a side-viewing electronic endoscope according to a first embodiment of the present invention;

FIG. 2 is a cross-sectional view taken along line A-A in FIG. 1, showing a maximum wide-angle state;

FIG. 3 is a view showing the state in which the maximum wide-angle state shown in FIG. 2 is switched to a maximum enlargement state, and an endo-therapy product raising base is raised;

FIG. 4 is a cross-sectional view taken along line B-B in FIG. 3;

FIG. 5 is a schematic perspective view of the endoscope; and

FIG. 6 is a plan view of a distal end section of a forward-viewing electronic endoscope according to a second embodiment of the invention.

### DETAILED DESCRIPTION OF THE INVENTION

Embodiments of the present invention will now be described with reference to the accompanying drawings.

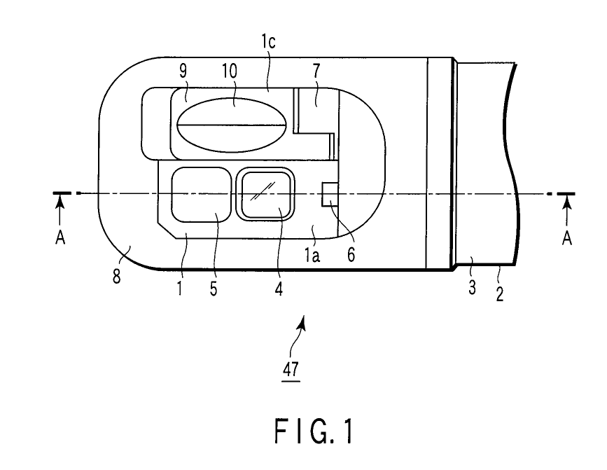

A first embodiment is described with reference to FIG. 1 to FIG. 5.

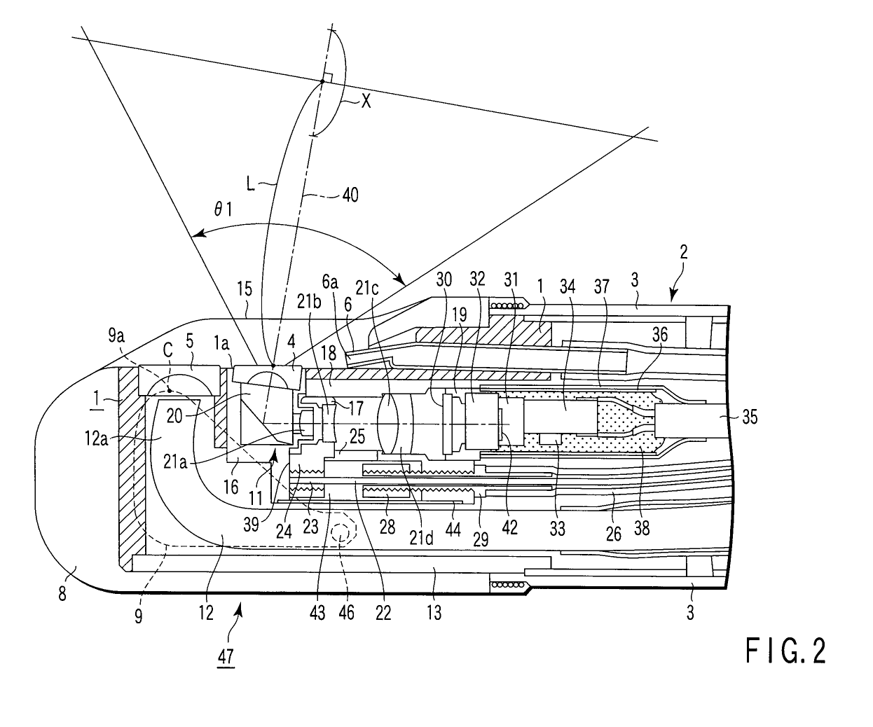

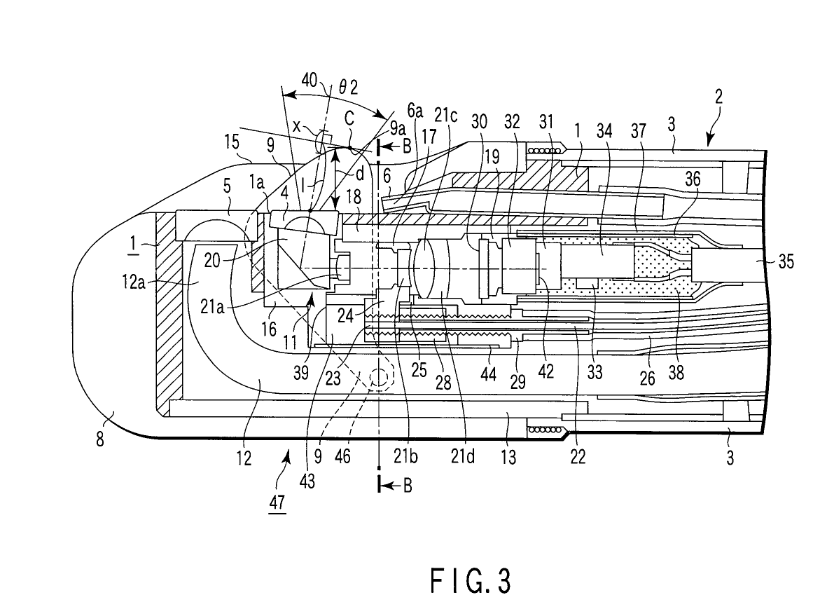

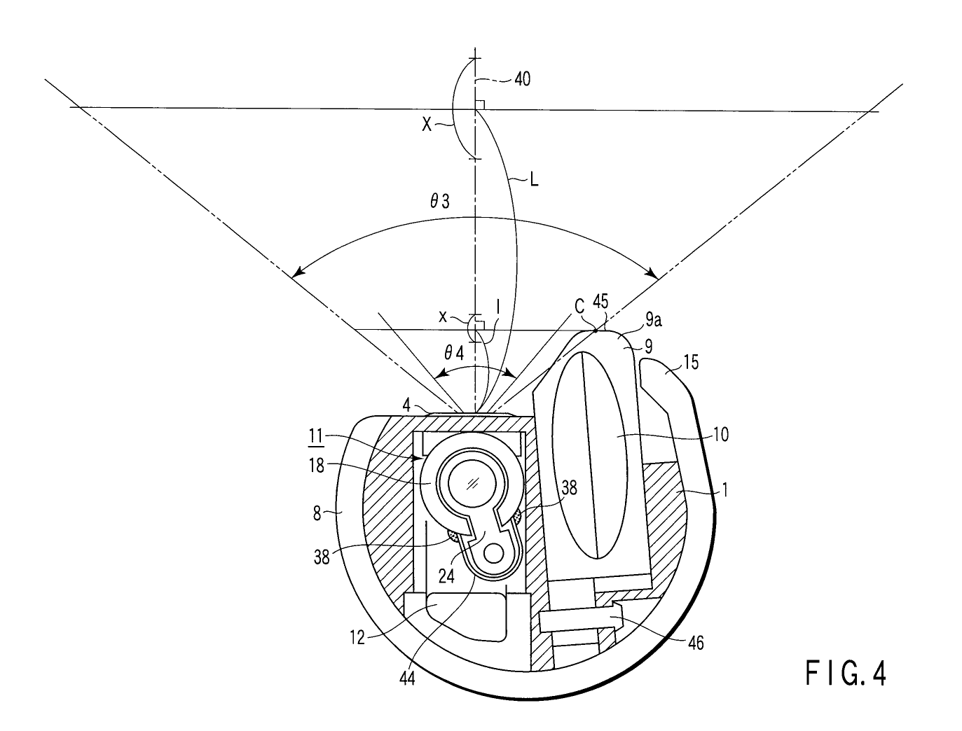

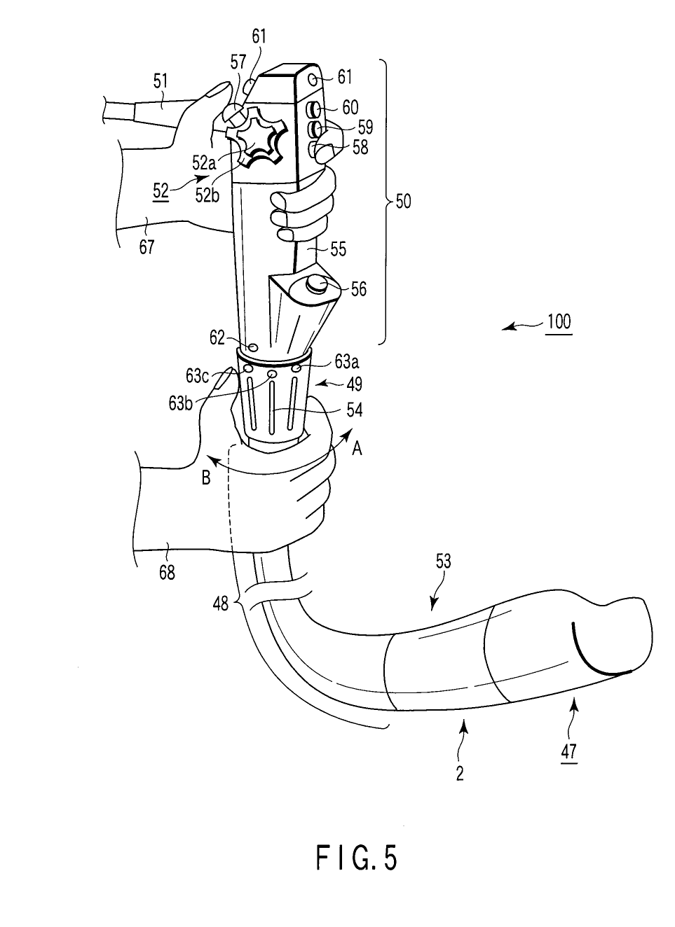

FIG. 1 is a plan view of a distal end section of a side-viewing electronic endoscope. FIG. 2 is a cross-sectional view taken along line A-A in FIG. 1, showing a maximum wide-angle state. FIG. 3 is a view showing the state in which the maximum wide-angle state shown in FIG. 2 is switched to a maximum enlargement state, and an endo-therapy product raising base is raised. FIG. 4 is a cross-sectional view taken along line B-B in FIG. 3. FIG. 5 is a schematic perspective view of the endoscope. In the description below, the term “wide-angle state” refers to the state of the distal end section body, in which an angle of view field for observation at a wide angle (hereinafter referred to as “wide-angle observation”) can be secured in order to guide (insert) the endoscope to, e.g. a surface of a mucous membrane, which is a part to be observed in a body cavity. The term “maximum wide-angle state” refers to the state of the distal end section body, in which a widest angle of view field can be secured at a time of wide-angle observation. The term “enlargement state” refers to the state of the distal end section body, in which an angle of view field for enlarging and observing the surface of the mucous membrane (hereinafter referred to as “enlargement observation”) can be secured. The term “maximum enlargement state” refers to the state of the distal end section body, in which an angle of view field for maximum enlargement can be secured at the time of enlargement observation.

An endoscope 100 in the present embodiment shown in FIG. 5 includes an insertion section 53 which, for example, has flexibility and is inserted in a body cavity. The endoscope 100 is a side-viewing electronic endoscope having a view field in a direction perpendicular to the direction of insertion of the insertion section 53. A distal end section 47 is disposed at a foremost end of the insertion section 53. A bending section 2 is disposed at an intermediate part of the insertion section 53. The distal end section 47 includes a distal end section body 1 shown in FIG. 1. The distal end section body 1 is formed of a metal such as stainless steel. A proximal end of the distal end section body 1 is coupled to a distal end of the bending section 2, as shown in FIG. 1 and FIG. 5. An outer surface of the bending section 2 is formed of, e.g. coating rubber 3, which is a soft, elastic member.

As shown in FIG. 1, a flat surface portion la is formed on a side surface of the distal end section body 1. In the flat surface portion la, there are disposed an illumination window 5 which transmits through light for illuminating, for example, a mucous membrane, which is a part to be observed, for example, in a body cavity; an observation window 4 which neighbors the illumination window 5 and on which reflective light of the light that has been transmitted through the illumination window 5 and reflected by the mucous membrane is made incident in order to observe the mucous membrane; an air-supply/water-supply nozzle 6 which is fixed to the distal end section body 1 in order to supply air or water to the mucous membrane; and an endo-therapy product projection port 7 from which an endo-therapy product (not shown) projects.

The observation window 4, illumination window 5 and air-supply/water-supply nozzle 6 are arranged in the longitudinal direction of the distal end section body 1. The observation window 4 is disposed more on the proximal end side of the distal end section body 1 than the illumination window 5. The air-supply/water-supply nozzle 6 is disposed more on the proximal end side of the distal end section body 1 than the observation window 4. In addition, as shown in FIG. 2, a nozzle face 6a of the air-supply/water-supply nozzle 6 is directed to the observation window 4 and illumination window 5.

The observation window 4 is a window which is disposed at the distal end section body 1 in order to observe a part to be observed which is located in a body cavity. The endo-therapy product projection port 7 is a projection port which is disposed near the observation window 4 and projects an endo-therapy product (not shown) that is inserted through the insertion section 53.

An endo-therapy product raising base 9 is provided in the vicinity of the endo-therapy product projection port 7. Specifically, the endo-therapy product raising base 9, which can be remotely operated to make oscillating movement in accordance with a raising operation on the proximal side of an operation section 50 (to be described later), is stored in the endo-therapy product projection port 7. For example, the endo-therapy product raising base 9 makes oscillating movement between a stored position, as indicated by a broken line in FIG. 2, where the endo-therapy product raising base 9 is stored in the distal end section body 1 (the endo-therapy product projection port 7), and a raised position, as shown in FIG. 3, where the endo-therapy product raising base 9 is projected (raised) from the flat surface portion 1a of the distal end section body 1, that is, from the endo-therapy product projection port 7. Specifically, a raising operation wire (not shown), for instance, is pushed and pulled in accordance with the raising operation on the proximal side of the operation section 50 (to be described later), and thereby the endo-therapy product raising base 9 is freely moved rotationally relative to the distal end section body 1 about a support shaft 46 shown in FIG. 2 to FIG. 4.

The endo-therapy product raising base 9 is oscillated in accordance with the raising operation, guides the endo-therapy product to a desired position, and projects and guides the endo-therapy product from the endo-therapy product projection port 7. In other words, when the endo-therapy product raising base 9 is stored in the stored position, the endo-therapy product raising base 9 raises the endo-therapy product and projects it from the endo-therapy product projection port 7.

A distal end side of an endo-therapy product insertion channel (not shown) is open at a proximal end side of the endo-therapy product raising base 9.

An endo-therapy product guide groove 10 for guiding the endo-therapy product from the distal end section body 1 to the outside is formed in a central part of the endo-therapy product raising base 9 in the longitudinal direction of the endo-therapy product raising base 9.

An electrically insulative distal end cap 8 is coated and fixed on the outer peripheral surface of the distal end section body 1, except the flat surface portion 1a where the observation window 4 and illumination window 5 are disposed, and an opening portion 1c of the endo-therapy product projection port 7. At the rear end of the distal end cap 8, the coating rubber 3 is watertightly attached to the distal end section body 1. In FIG. 2, the distal end cap 8 is configured to be fixed to the distal end section body 1. However, the structure of the distal end cap 8 is not limited to this, and the distal end cap 8 may be configured to be detachably attached to the distal end section body 1.

As shown in FIG. 2, in the distal end section body 1, the observation window 4 and an objective optical system unit 11 are disposed as one unit. The objective optical system unit 11 is built in the observation window 4. The objective optical system unit 11 is disposed under the flat surface portion 1a. The focal length of the objective optical system unit 11 is variable at the time of observing a part which is to be observed.

A light guide fiber 12 is disposed under the objective optical system unit 11 in the same direction as the objective optical system unit 11. The light guide fiber 12 transmits (guides) light, which is emitted from a light source device (not shown), to the illumination window 5. A distal end portion 12a of the light guide fiber 12 is bent so as to be connected to the illumination window 5 from under the objective optical system unit 11. A cover member 13, which covers the light guide fiber 12 at the distal end section body 1, is disposed on the outside of the light guide fiber 12. The cover member 13 is bonded to the distal end section body 1. At the rear end of the distal end cap 8, the coating rubber 3 is watertightly attached to the cover member 13.

The broken line in FIG. 2 shows the stored state of the endo-therapy product raising base 9 in the distal end section body 1. In this stored state, the endo-therapy product raising base 9 is positioned in a fallen state. In this state, a distal end 9a, which is a remotest point C from the support shaft 46, is positioned below a side wall 15 of the distal end cap 8.

Next, referring to FIG. 2 and FIG. 3, the objective optical system unit 11 is described in detail.

The objective optical system unit 11, which is a single unit, is composed of four small units, namely, a first lens frame 16, a second lens frame 17, a third lens frame 18 and a fourth lens frame 19. The first lens frame 16, second lens frame 17, third lens frame 18 and fourth lens frame 19 are arranged from the distal end side of the objective optical system unit 11 in the axial direction of the objective optical system 11.

In the first lens frame 16, there are disposed the observation window 4; a prism 20 which reflects the reflective light transmitting through the observation window 4 and varies the progress of the light (i.e. deflects an optical axis 40); and a convex lens that is a first lens 21a which passes the reflective light that is reflected by the prism 20. A stopper surface 39, which restricts a wide-angle-side movement range (to be described later) of the second lens frame 17, is integrally formed on the first lens frame 16. The observation window 4, prism 20 and first lens 21a are successively arranged in the direction of travel of the reflective light.

In the second lens frame 17, there are disposed a concave lens which is a second lens 21b which transmits through the reflective light that has transmit through the first lens 21a; and an arm portion 24 which holds the second lens 21b and is advancible/retreatable along the axis direction of the objective optical system unit 11. A zoom operation wire 22 is fixed to the arm portion 24. The zoom operation wire 22 advances/retreats the arm portion 24 along the axis of the objective optical system unit 11, thereby advancing/retreating the second lens 21b along the optical axis direction (i.e. the axial direction of the objective optical system unit 11). Specifically, the zoom operation wire 22 is fixed to the arm portion 24 at a distal end 23 of the zoom operation wire 22. The arm portion 24 extends in a direction in which the light guide fiber 12 is disposed. As shown in FIG. 2, the arm portion 24 touches on the stopper surface 39, thereby restricting the wide-angle-side movement range. In other words, the arm portion 24 touches on the stopper surface 39, thereby setting angles of view field, θ1 and θ3, in the maximum wide-angle state shown in FIG. 2 and FIG. 4. The second lens 21b advances/retreats in the optical axis direction, and thereby the focal length of the objective optical system unit 11 is varied.

In the third lens frame 18, a slit 25, along which the arm portion 24 slides, is formed in the direction of the axis of the third lens frame 18 (i.e. the axis of the objective optical system unit 11) over a range between a distal end and an intermediate part of the third lens frame 18.

In addition, in the third lens frame 18, at an intermediate part of the third lens frame 18, there are disposed a convex lens which is a third lens 21c for transmitting through the reflective light transmitting through the second lens 21b, and a concave lens which is a fourth lens 21d which transmits through the reflective light transmitting through the third lens 21c. The third lens 21c is disposed in front of the second lens 21b in the direction of travel of the reflective light, that is, on the proximal end side of the objective optical system unit 11 along the axis of the objective optical system unit 11. In addition, the fourth lens 21d is disposed in front of the third lens 21c in the direction of travel of the reflective light.

In the third lens frame 18, a mouthpiece 29 is fixed to a proximal end of the third lens frame 18 in a manner to penetrate in the axial direction of the objective optical system unit 11. A guide member 26, which guides the zoom operation wire 22 to the arm portion 24, is attached to the mouthpiece 29. A stopper 28, which restricts the enlargement-side movement range of the second lens frame 17, is adjustably fixed on the distal end side of the mouthpiece 29. Specifically, the arm portion 24 touches on the stopper 28, and thereby the angles of view field, θ2 and θ4, which will be described later, in the maximum enlargement state shown in FIG. 3 and FIG. 4 are set.

In the fourth lens frame 19, there are disposed an optical filter 30 which is positioned more on the proximal end side of the objective optical system unit 11, than the fourth lens 21d, in the axial direction of the objective optical system unit 11, and which shades, e.g. infrared light, and transmits through visible light; and a cover glass 32 which is positioned more on the proximal end side of the objective optical system unit 11, than the optical filter 30, in the axial direction of the objective optical system unit 11, and which is formed integral with an image pickup element 31 such as a CCD. The image pickup element 31 receives the reflective light, which is transmitted through the optical filter 30, on its light-receiving surface 42, and converts the reflective light to an electric signal that is a video signal.

The above-described image pickup element 31, an electric board 34 which is connected to the image pickup element 31, and a signal cable 35 which is connected to the electric board 34 are successively disposed from the distal end side of the objective optical system unit 11 along the axial direction of the objective optical system unit 11.

An electric component 33 is mounted on the electric board 34, and the electric board 34 amplifies the electric signal. The signal cable 35 transmits the electric signal to a video processor (not shown).

The outside (outer periphery) of the image pickup element 31 and electric board 34 is protected by a thin metal plate 36 having a complete circumferential shape. The outside of the metal plate 36 is coated with an electrically insulative tube 37. An adhesive 38 is filled between the electric board 34, signal cable 35 and metal plate 36. Thereby, the fourth lens frame 19, image pickup element 31, electric board 34, signal cable 35 and metal plate 36 are constituted as one unit.

The first lens frame 16 is bonded and fixed to the distal end face of the second lens frame 17.

The arm portion 24 of the second lens frame 17 advances/retreats along the inner surface of the third lens frame 18 in the axial direction of the objective optical system unit 11 by a remote control by means of the zoom operation wire 22. At this time, in the second lens frame 17, the arm portion 24 slides along the slit 25 and moves toward the stopper surface 39 in the case where wide-angle observation is performed as illustrated in FIG. 2, as described above. In the case where enlargement observation is performed, as illustrated in FIG. 3, the arm portion 24 slides along the slit 25 and moves toward the stopper 28. In short, the second lens frame 17 slides along the slit 25 and advances/retreats between the stopper surface 39 and the stopper 28. Thereby, the objective optical system unit 11 varies the focal length thereof. Thus, the endoscope 100 according to the present embodiment is an endoscope with a focal length varying function, in which the focal length can be varied.

The fourth lens frame 19 is optically aligned with, and bonded and fixed to, the third lens frame 18.

A dot-and-dash line in FIG. 2 indicates an optical axis 40. The optical axis 40 is reflected by the prism 20 and made substantially parallel with the axial direction of the objective optical system unit 11 within the objective optical system unit 11, and is focused on the light-receiving surface 42.

A subject image focused on the light-receiving surface 42 is converted to an electric signal by the image pickup element 31, and then amplified and transmitted to the video processor (not shown) via the signal cable 35. Further, the subject image is subjected to signal processing in the video processor and is displayed as an observation image on a monitor (not shown).

A space 43, in which the arm portion 24 is advanced/retreated by the zoom operation wire 22, is shielded by a cover member 44 from the outside of the objective optical system unit 11. As shown in FIG. 4, an adhesive 38 is coated on end portions of the cover member 44 over the entire region. The cover member 44 is bonded to the objective optical system unit 11 by the adhesive 38. Thereby, the cover member 44 prevents foreign matter (i.e. moisture, water drops, dust) from entering the objective optical system unit 11 including the space 43 from the outside of the objective optical system unit 11.

The angle (angle of view field) θ1 in FIG. 2 indicates the angle of view field in the maximum wide-angle state, as described above. At this time, the arm portion 24 touches on the stopper surface 39. Assume now that the depth of focus of the objective optical system unit 11 in the maximum wide-angle state is X, and the best focal length of the objective optical system unit 11 is a length L from the observation window 4. In short, the best focus position of the objective optical system unit 11 is a position apart from the observation window 4 by the length L. The whole lens structure is not limited to that shown in FIG. 2.

The angle (angle of view field) θ2 in FIG. 3 indicates the angle of view field in the maximum enlargement state, as described above. At this time, the arm portion 24 touches on the stopper 28. The direction of the optical axis 40 at this time is the same as in the state shown in FIG. 2, but the angle (angle of view field) θ2 in FIG. 3 is less than the angle of view field, θ1, in FIG. 2 (θ1>θ2).

Assume now that the depth of focus of the objective optical system unit 11 in the maximum enlargement state is x, and the best focal length of the objective optical system unit 11 is a length 1 from the observation window 4. In short, the best focus position of the objective optical system unit 11 is a position apart from the observation window 4 by the length 1. The depth of focus, x, is less than the depth of focus, X (X>x). The best focal length 1 is less than the best focal length L (L>1).

In FIG. 2 and FIG. 3, the endo-therapy product raising base 9 is raised or fallen independently from the arm portion 24, not associate with the arm portion 24 that moves between the stopper 28 and the stopper surface 39.

In the present embodiment, when the endo-therapy product raising base 9 moves rotationally about the support shaft 46 and the endo-therapy product raising base 9 projects from the distal end section body 1 by a predetermined amount, a predetermined part of the endo-therapy product raising base 9 is set to approximately correspond to the best focus position of the objective optical system unit 11. In other words, when the endo-therapy product raising base 9 is raised and the distal end 9a is projected from the distal end section body 1 by a predetermined amount, this predetermined amount in this embodiment is set to approximately correspond to the best focal length 1 of the objective optical system unit 11.

For example, as shown in FIG. 3, when the endo-therapy product raising base 9 is projected from the distal end section body 1 by the maximum amount, the predetermined part of the endo-therapy product raising base 9 is set to approximately correspond to the best focus position of the objective optical system unit 11 at the time of maximum enlargement (i.e. the maximum enlargement state). In other words, when the endo-therapy product raising base 9 is raised to a maximum degree, the above-described predetermined amount is set to approximately correspond to the best focal length 1 of the objective optical system unit 11 in the maximum enlargement state.

Specifically, when the endo-therapy product raising base 9, which is raised as shown in FIG. 3, is projected in the longitudinal axis direction of the distal end section body 1, a distance d from the observation window 4 to the remotest point C (distal end 9a) is so preset as to approximately correspond to the best focal length of the objective optical system unit 11, for example, as shown in FIG. 3. In other words, the distance d is the length from the observation window 4 to the distal end 9a that is the remotest point C in the height direction which is substantially perpendicular to the flat surface portion 1a. The observation window 4 is a reference plane in the height direction. For example, in the case where the observation window 4 and the flat surface portion 1a are substantially in the same plane, the distance d is the length in the height direction from the flat surface portion 1a to the distal end 9a. Since the endoscope of the present embodiment is of the side-viewing type, the distal end 9a projects from the endo-therapy product projection port 7 in the flat surface portion 1a, that is, from the side surface of the distal end section body 1.

As has been described above, when the endo-therapy product raising base 9 is raised, the distance d is the length from the observation window 4 to the distal end 9a in the direction that is substantially perpendicular to the side surface, and is the above-described predetermined amount, and is so set as to approximately correspond to the best focal length of the objective optical system unit 11.

In FIG. 4, θ3 indicates an angle of view field in a lateral direction in the maximum wide-angle state as shown in FIG. 2, and θ4 indicates an angle of view field in the lateral direction in the maximum enlargement state as shown in FIG. 3. The optical axis 40 does no vary in these states.

FIG. 4 shows the relationship between the depth of focus, X, and the best focal length L shown in FIG. 2, and the depth of focus, x, and the best focal length 1 shown in FIG. 3. As shown in FIG. 4, if the endo-therapy product raising base 9 is raised to the maximum degree at the angle of view field, θ3, of the observation window 4 in the maximum wide-angle state, the endo-therapy product raising base 9 partly enters (overlaps) the view field of observation (the angle of view field θ3) and is visible. However, at the angle of view field, θ4, of the observation window 4 in the maximum enlargement state, even if the endo-therapy product raising base 9 is raised to the maximum degree, the endo-therapy product raising base 9 does not enter (overlap) the view field of observation (the angle of view field θ4) and is invisible. In this manner, even when the endo-therapy product raising base 9 is raised to the maximum degree, the endo-therapy product raising base 9 is positioned outside the angle of view field, θ4.

The arm portion 24, as described above, extends toward the light guide fiber 12, and is disposed with an inclination to the center axis of the distal end section body 1 of the endoscope 100.

The cover member 44 has a substantially U shape as shown in FIG. 4, and is bonded to the objective optical system unit 11 by the adhesive 38, as described above, thereby preventing foreign matter (i.e. moisture, water drops, dust) from entering the objective optical system unit 11. In addition, as shown in FIG. 2 to FIG. 4, the endo-therapy product raising base 9 is disposed so as to project from the side surface of the distal end section body 1.

As regards the endo-therapy product raising base 9 that is raised to the maximum degree as shown in FIG. 4, it is preferable that a distal-end edge line 45 at the distal end 9a be substantially parallel to the observation window 4, as shown in FIG. 4. Thereby, the distal-end edge line 45 comes in surface-contact, and not point-contact, with the mucous membrane. When the raised endo-therapy product raising base 9 comes in contact with the mucous membrane, the position of the endo-therapy product raising base 9, relative to the mucous membrane, is stabilized.

The endo-therapy product raising base 9 has the support shaft 46 which is an oscillation shaft for oscillation between the above-described stored position and the raised position, and is a movement rotationally shaft for movement rotationally relative to the distal end section body 1. The endo-therapy product raising base 9 is held to the distal end section body 1 so as to movable rotationally about the support shaft 46.

Next, referring to FIG. 5, the endoscope 100 is described in brief.

The endoscope 100 includes an insertion section 53 which is inserted, for example, in a body cavity of a patient; a zoom operation section 49 which is coupled to a proximal end portion of the insertion section 53 and performs a zoom operation; and the operation section 50 which is coupled to the zoom operation section 49 and operates the insertion section 53.

The insertion section 53 includes a flexible tube portion (flexible insertion tube portion) 48, a bending section 2 and the distal end section 47, in the named order from the zoom operation section 49 side. Specifically, the zoom operation section 49 is coupled to a proximal end of the flexible tube portion (flexible insertion tube portion) 48. A distal end of the flexible tube portion 48 is coupled to a proximal end of the bending section 2. A distal end of the bending section 2 is coupled to a proximal end of the distal end section 47.

The bending section 2 is bent in four directions, namely, upward, downward, leftward and rightward directions, by the operation of a bending operation knob 52 (to be described later). Thereby, the distal end section 47, which is coupled to the bending section 2, bends in the same direction as the bending section 2.

The operation section 50 is provided with a hold portion 55 which is grasped by a surgeon, and a bending operation knob 52 which bends the bending section 2.

A proximal end portion of a universal cord 51 is coupled to the hold portion 55. A distal end portion of the universal cord 51 is connected to a light source device (not shown) which emits light, and to a connector section (not shown) for connection to a video processor.

The light guide fiber 12, for instance, is passed through the universal cord 51, flexible tube portion 48 and bending section 2. Light that is emitted from the light source device is guided via the light guide fiber 12 and is radiated from the illumination window 5 onto a part to be observed. The signal cable 35, for instance, is passed through the flexible tube portion 48 and bending section 2.

The bending operation knob 52 is provided with a right-and-left bending operation knob 52a for bending the bending section 2 in a right-and-left direction, and an up-and-down bending operation knob 52b for bending the bending section 2 in an up-and-down direction. A right-and-left bending operation mechanism (not shown), which is driven by the right-and-left bending operation knob 52a, is connected to the right-and-left bending operation knob 52a. An up-and-down bending operation mechanism (not shown), which is driven by the up-and-down bending operation knob 52b, is connected to the up-and-down bending operation knob 52b. The right-and-left bending operation mechanism and the up-and-down bending operation mechanism are disposed in the operation section 50, and are connected to proximal end portions of operation wires (not shown) for bending the bending section 2 in the upward, downward, leftward and rightward directions.

The operation section 50 is provided with an endo-therapy product insertion portion 56 which is a proximal-side opening portion of an endo-therapy product insertion channel (not shown) communicating with the endo-therapy product projection port 7; an endo-therapy product raising base operation lever 57 for raising the endo-therapy product raising base 9 by a remote operation using, e.g. a raising operation wire; an air-supply/water-supply button 58 for supplying air or water from the air-supply/water-supply nozzle 6 toward the observation window 4, etc.; a suction button 59 for a suction operation for suction from the distal end section 47 via a suction channel (not shown) which is branched from the endo-therapy product insertion channel (not shown) in the operation section 50; a release switch 60 which stores an observation image in a memory section (not shown); and an image process switch 61 for an image process of the observation image.

The zoom operation section 49 is provided with a zoom ring 54 which is movable rotationally in a circumferential direction about the insertion direction of the insertion section 53. If the zoom ring 54 is moved rotationally in a direction A (counterclockwise as viewed from the hold portion 55 side), the zoom operation wire 22 is pulled from the state shown in FIG. 2 and the second lens frame 17 moves (retreats) toward the stopper 28. If the second lens frame 17 touches on the stopper 28, as shown in FIG. 3, maximum enlargement observation is enabled.

If the zoom ring 54 is moved rotationally in a direction B (clockwise as viewed from the hold portion 55 side), the zoom operation wire 22 is pushed from the state shown in FIG. 3 and the second lens frame 17 moves (advances) toward the stopper surface 39. If the second lens frame 17 touches on the stopper surface 39, as shown in FIG. 2, maximum wide-angle observation is enabled.

The zoom ring 54 is provided with indices 63a, 63b and 63c which indicate enlargement magnification ratios by numerals, etc. The operation section 50 is provided with an index 62 in the vicinity of the zoom operation section 49, for alignment with the indices 63a, 63b and 63c. Specifically, if the zoom ring 54 is moved rotationally and any one of the indices 63a, 63b and 63c is aligned with the index 62, the monitor (not shown) of, e.g. 14 inches displays an observation image with a magnification corresponding to the index 63.

A cam mechanism (not shown) is built in the zoom operation section 49. The cam mechanism converts moving rotational movement of the zoom ring 54 to a linear advancing/retreating movement of the zoom operation wire 22. In the present embodiment, the structures are not necessarily limited to the above.

Next, a description is given of an operation method at a time of close-up enlargement observation.

To begin with, if the endo-therapy product raising base 9 is not raised and the zoom ring 54 is moved rotationally in the direction B, with the index 63a being aligned with the index 62, the zoom operation wire 22 is pushed and the second lens frame 17 advances and touches on the stopper surface 39, as shown in FIG. 2. Thus, the distal end section 47 is set in the maximum wide-angle state. In this state, the endoscope 100 captures an image of, for example, the mucous membrane (subject), which is the part to be observed in the body cavity, by means of the image pickup element 31 via the observation window 4. The captured image is displayed on the monitor, and the mucous membrane is probed.

Subsequently, the bending section 2 is bent by the bending operation knob 52 and the operation wire (not shown), and the insertion section 53 is pushed and pulled. Thus, the distal end section 47 is moved close to the mucous membrane.

At this time, the mucous membrane is located outside the depth of focus, X, of the objective optical system unit 11. Consequently, the image displayed on the monitor is not in focus.

Thereafter, if the zoom ring 54 is moved rotationally in the direction A and the index 63c is aligned with the index 62 as shown in FIG. 5, the zoom operation wire 22 is pulled and the second lens frame 17 retreats and touches on the stopper 28, as shown in FIG. 3. Thus, the distal end section 47 is set in the maximum enlargement state. Then, the position of the mucous membrane falls within the range of the depth of focus, x, or suddenly falls out of the range of the depth of focus x. Consequently, the image of the mucous membrane is clearly visible at one time and blurs at other time, and stable observation of the mucous membrane cannot be performed.

In this state, if the endo-therapy product raising base operation lever 57 of the operation section 50 is operated and the endo-therapy product raising base 9 is remotely operated by the raising operation wire, etc., the endo-therapy product raising base 9 is raised to the maximum degree. Further, if the insertion section 53 is pushed and pulled, the distal end 9a, which is the remotest point C of the endo-therapy product raising base 9 shown in FIG. 3, comes in contact with the mucous membrane. At this time, the distance d is the above-described approximately best focal length.

Thus, the distance between the mucous membrane and the objective optical system unit 11 at the time of close-up enlargement observation can easily be set at the approximately best focal length, which is approximately equal to the best focal length L, by putting the distal end 9a in contact with the mucous membrane.

As has been described above, light is emitted from the light source device, as described above. The light is made incident on the connector section, and is radiated to the part to be observed from the illumination window 5 via the light guide fiber 12 which is passed through the universal cord 51, flexible tube portion 48 and bending section 2. Reflective light from the illuminated part to be observed is made incident on the observation window 4 and is reflected by the prism 20. The reflective light then transmits through the first lens 21a, second lens 21b, third lens 21c, fourth lens 21d, optical filter 30 and cover glass 32. Infrared light of the reflective light is shaded by the optical filter 30, and only the visible light of the reflective light is transmitted through the optical filter 30. The reflective light (visible light) transmitting through the optical filter 30 is transmitted through the cover glass 32, is focused on the light-receiving surface 42, and is converted to an electric signal by the image pickup element 31. The electric signal is amplified by the electric board 34, and is output to the monitor (not shown) via the signal cable 35. Thus, the electric signal is displayed as a subject image on the monitor. An image of the mucous membrane, which is clear and in focus without blurring, is displayed in enlarged scale on the monitor. Thereby, the endoscope 100 performs enlargement observation of the mucous membrane in a stable in-focus state.

As has been described above, in the present embodiment, in order to secure the best focal length L, there is no need to attach a distal end hood to the distal end section 47, or to dispose a built-in advancing/retreating mechanism for advancing/retreating the distal end hood to associate with the variation of the focal length of the objective optical system unit 11. In the present embodiment, in order to secure the best focal length L, it is necessary to provide only the endo-therapy product raising base 9 which raises the endo-therapy product, the approximately best focal length of the endo-therapy product raising base 9 being set in advance. Thereby, in the present embodiment, when close-up enlargement observation of the part to be observed is performed, there is no need to use a dedicated structural member for securing the best focal length. Only by the operation of raising the endo-therapy product raising base 9 which is mounted in advance in order to raise the endo-therapy product, the distance between the mucous membrane and the objective optical system unit 11 can easily be set at the approximately best focal length, even when the focal length of the objective optical system unit 11 is varied.

In the present embodiment, the manufacturing cost is low since a dedicated structure for securing the best focal length is made needless by using the pre-mounted endo-therapy product raising base 9 also for securing the best focal length. Furthermore, since the distal end hood is not mounted, an increase in diameter of the distal end section can be prevented.

As shown in FIG. 4, since the endo-therapy product raising base 9 is inclined toward the center axis of the distal end section 47, the best focal length can be finely adjusted. Therefore, in the present embodiment, the focus state can also be finely adjusted.

At the time of maximum enlargement, as shown in FIG. 4, the endo-therapy product raising base 9 does not enter the field of view for observation (θ4) and is located outside the field of view for observation (θ4). Thus, in the present embodiment, the field of view for observation is prevented from being eclipsed by the endo-therapy product raising base 9, and the occurrence of a problem with the observation can be prevented.

In the present embodiment, as shown in FIG. 4, the arm portion 24 is disposed with an inclination toward the center axis of the distal end section body 1. Thereby, in this embodiment, the zoom operation wire 22 can be biased in position toward the center of the insertion section 53, and damage to the zoom operation wire 22 due to other built-in components at the time of bending operation can be minimized. In the embodiment, the zoom operation wire 22 is slightly advanced/retreated only by the bending operation. However, by biasing the position of the zoom operation wire 22 toward the center, the amount of advancement/retreat can be made less than in the case where the zoom operation wire 22 is positioned at the peripheral part of the distal end section body 1.

In the present embodiment, the cover member 44 prevents entrance of foreign matter (i.e. moisture, water drops, dust). Thereby, in the embodiment, it is possible to prevent foreign matter (i.e. moisture, water drops, dust) from entering the field of view for observation and becoming an obstacle to observation.

In the present embodiment, the distal-end edge line 45 is put in surface-contact with the part to be observed. Thereby, in the embodiment, the distal end 9a can more easily be put in contact with the mucous membrane than in the case of point contact, and displacement of the distal end section 47 can be prevented at the time of observing the part to be observed. In short, in the embodiment, the best focal length can be stabilized.

In this embodiment, for example, as shown in FIG. 5, the hold portion 55 is grasped by the user's left hand 67, and the zoom ring 54 is moved rotationally by the right hand 68. Thereby, the moved rotational operation of the zoom ring 54 can finely be adjusted.

In the present embodiment, in order to prevent the distal end section 47 from moving in the body cavity, for example, at a time of an inspection, it is preferable to make the user to hold the insertion section 53 by the right hand 68, which is disposed more on the front side in the direction of insertion than the zoom ring 54. Thereby, in the embodiment, since the zoom ring 54 is positioned immediately near the right hand 68, the zoom operation can easily be performed. In addition, after the zoom operation, the right hand 68 can immediately be moved back to hold the insertion section 53.

The index 63 may be a printed mark on the zoom ring 54 or may be a seal or the like, which can arbitrarily be put on the body of the zoom ring 54. In the embodiment, for example, if the position of attachment of the index 63, which is a seal, is adjustable, the position of attachment of the index 63 can be adjusted even when the zoom operation wire 22 extends due to long-time use. Thereby, a correct enlargement magnification index can be put on a desired position. In addition, in the present embodiment, if the index 63 is a seal or the like, enlargement magnification indices corresponding to users can be attached.

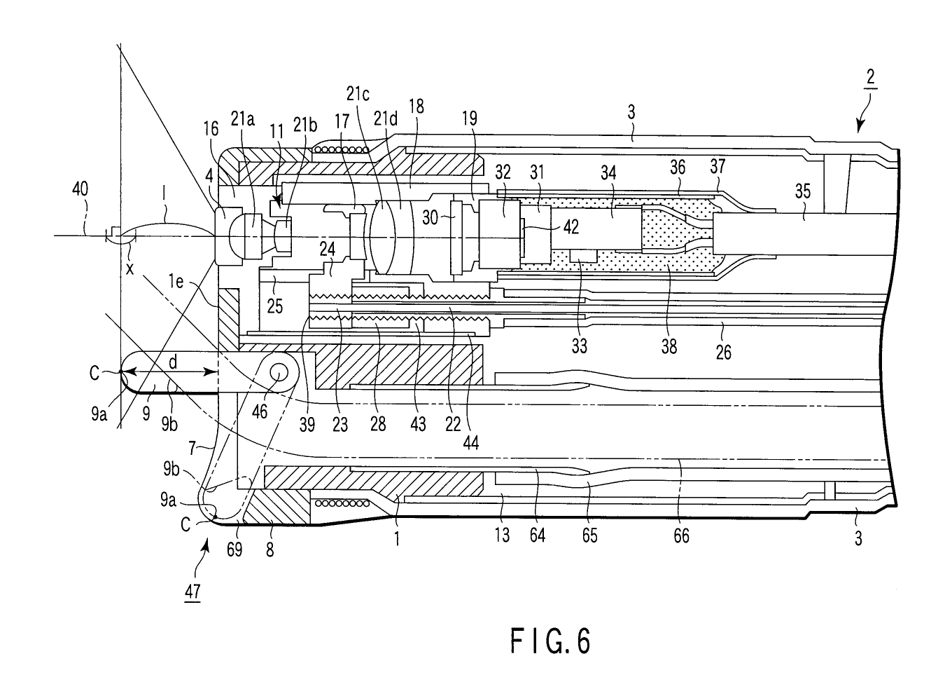

Next, a second embodiment of the present invention is described with reference to FIG. 6. The same structural parts as in the first embodiment are denoted by the same reference numerals as in the first embodiment, and a description is omitted. FIG. 6 is a plan view of a distal end section of a forward-viewing electronic endoscope in a maximum enlargement state.

The above-described first embodiment relates to the side-viewing electronic endoscope, whereas the present embodiment relates to a forward-viewing electronic endoscope. Accordingly, a front surface 1e of the distal end section body 1 is provided with an endo-therapy product raising base 9 which projects from the front surface 1e when the endo-therapy product raising base 9 is moved rotationally about the support shaft 46; an observation window 4; an endo-therapy product projection port 7; an illumination window 5 (not shown in FIG. 6); and an air-supply/water-supply nozzle 6.

The endo-therapy product raising base 9 has a raising surface 9b for raising an endo-therapy product 66 when the endo-therapy product raising base 9 is moved rotationally about the support shaft 46 and raised. The endo-therapy product raising base 9 shown in FIG. 6 in the present embodiment is smaller in size than the endo-therapy product raising base 9 shown in FIG. 2 in the first embodiment. Like the first embodiment, depiction of a raising operation wire for raising the endo-therapy product raising base 9 is omitted.

An endo-therapy product insertion channel 65 is connected to the distal end section body 1 via a connection member 64.

A prism 20 is not built in the objective optical system unit 11. The observation window 4 is disposed behind the first lens 21a in the direction of travel of reflective light. The objective optical system unit 11, bending section 2, flexible tube portion 48, zoom operation section 49 and operation section 50 have the same structures as in the above-described first embodiment.

When the endo-therapy product 66 is raised, the endo-therapy product 66 is put in contact with the raising surface 9b by the endo-therapy product raising base 9 that is moved rotationally, and the endo-therapy product 66 is raised as indicated by a two-dot-and-dash line in FIG. 6. The raised position of the endo-therapy product raising base 9, which is raised to the maximum degree, is indicated by a solid line in FIG. 6. The stored position of the endo-therapy product raising base 9, which is lowered to the maximum degree and stored in the distal end section body 1, is indicated by a two-dot-and-dash line in FIG. 6. At this time, the endo-therapy product raising base 9 does not project from the distal end section 47, but it may slightly project therefrom.

In FIG. 6, assume that the depth of focus of the objective optical system unit 11 is x, and the best focal length of the objective optical system unit 11 is set at a position away from the observation window 4 by a length 1.

A distance d, like the first embodiment, is preset at an approximately best focal length of the objective optical system unit 11, which is approximately equal to the best focal length 1. Since the endoscope of the present embodiment is of the forward-viewing type, the distal end 9a projects from the endo-therapy product projection port 7 in the front surface 1e. Accordingly, the distance d is the length from the observation window 4 to the distal end 9a in the height direction (the right-and-left direction in FIG. 6; the axial direction of the objective optical system unit 11) which is substantially perpendicular to the front surface 1e. The observation window 4 is a reference plane in the height direction. For example, in the case where the observation window 4 and the front surface 1e are substantially in the same plane, the distance d is the length in the height direction from the front surface 1e to the distal end 9a.

As described above, when the endo-therapy product raising base 9 is raised to the maximum degree in the maximum enlargement state, the distance d is the length from the observation window 4 to the distal end 9a in the direction substantially perpendicular to the front surface 1e, and is set to approximately correspond to the best focal length of the objective optical system unit 11.

The operation method at a time of close-up enlargement observation in the present embodiment is substantially the same as that in the above-described first embodiment, so a description is omitted here.

Thus, in the present embodiment, even with the forward-viewing electron endoscope, substantially the same advantageous effects as in the first embodiment can be obtained.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope with a focal length varying function, comprising:
an observation window which is disposed on a side of a distal end section of an insertion section;
an objective optical system which is built in the observation window and is capable of varying a focal length thereof; and
an endo-therapy product raising base which is disposed in a vicinity of an endo-therapy product projection port of the distal end section and is oscillatable between a stored position where the endo-therapy product raising base is stored in the distal end section and a raised position where the endo-therapy product raising base is projected from the distal end section in accordance with a raising operation on a proximal side,
wherein at a time of a state in which the endo-therapy product raising base is projected from the distal end section side by a predetermined amount, a predetermined part of the endo-therapy product raising base is set to approximately correspond to a best focus position of the objective optical system.

2. The endoscope with a focal length varying function, according to claim 1, wherein the endoscope is a side-viewing endoscope in which the observation window is disposed on a side surface of the distal end section of the insertion section, and the endo-therapy product raising base is disposed in a manner to project from the side surface of the distal end section of the insertion section.

3. The endoscope with a focal length varying function, according to claim 2, wherein at a time of a state in which the endo-therapy product raising base is projected from the distal end section to a maximum degree, the predetermined part of the endo-therapy product raising base is set to approximately correspond to a best focus position of the objective optical system at a time of maximum enlargement.

4. The endoscope with a focal length varying function, according to claim 1, wherein at a time of a state in which the endo-therapy product raising base is projected from the distal end section to a maximum degree, the predetermined part of the endo-therapy product raising base is set to approximately correspond to a best focus position of the objective optical system at a time of maximum enlargement.

5. An endoscope with a focal length varying function, comprising:
a distal end section of an insertion section which is inserted in a body cavity;
an observation window which is disposed on the distal end section and configured to observe a part to observed, which is located in the body cavity;
an objective optical system which is built in the observation window and is capable of varying a focal length thereof at a time of observing the part to be observed;
an endo-therapy product projection port which is disposed in a vicinity of the observation window in the distal end section and configured to project an endo-therapy product which is passed through the insertion section; and
an endo-therapy product raising base which is disposed in the endo-therapy product projection port, is oscillatable between a stored position where the endo-therapy product raising base is stored in the distal end section and a raised position where the endo-therapy product raising base is projected from the distal end section in accordance with a raising operation on a proximal side, and raises the endo-therapy product when the endo-therapy product raising base is disposed in the stored position, thereby projecting the endo-therapy product from the endo-therapy product projection port,
wherein when the endo-therapy product raising base is raised and projected from the distal end section by a desired length, the length is set to approximately correspond to a best focal length of the objective optical system.

6. The endoscope with a focal length varying function, according to claim 5, wherein when the endo-therapy product raising base is raised from the distal end section to a maximum degree and an image of the part to be observed is observed in maximum enlargement, the length is set to approximately correspond to the best focal length of the objective optical system.

7. The endoscope with a focal length varying function, according to claim 5, wherein the endo-therapy product raising base has an oscillation shaft for oscillation.

8. The endoscope with a focal length varying function, according to claim 5, wherein when the endo-therapy product raising base is raised from the distal end section to a maximum degree and an image of the part to be observed is observed in maximum enlargement, the endo-therapy product raising base is disposed outside of a field of view for observation of the observation window.

9. The endoscope with a focal length varying function, according to claim 5, wherein when the endo-therapy product raising base is raised from the distal end section to a maximum degree, a distal end of the endo-therapy product raising base is substantially parallel to the observation window and comes in surface-contact with the part to be observed.

10. The endoscope with a focal length varying function, according to claim 5, wherein the endoscope is of a side-viewing type in which the observation window is disposed on a side surface of the distal end section, and the endo-therapy product raising base is disposed in a manner to be raised from the side surface.

11. The endoscope with a focal length varying function, according to claim 10, wherein when the endo-therapy product raising base is raised from the side surface, the length is a length from the observation window to a distal end of the raised endo-therapy product raising base in a direction substantially perpendicular to the side surface.

12. The endoscope with a focal length varying function, according to claim 5, wherein the endoscope is of a forward-viewing type in which the observation window is disposed on a front surface of the distal end section, and the endo-therapy product raising base is disposed in a manner to be raised from the front surface.

13. The endoscope with a focal length varying function, according to claim 12, wherein when the endo-therapy product raising base is raised from the front surface, the length is a length from the observation window to a distal end of the raised endo-therapy product raising base in a direction substantially perpendicular to the front surface.
